(19) United States
(12) Patent Application Publication
(10) Pub. No.: US 2010/0178249 A1
(43) Pub. Date: Jul. 15, 2010

(54) IMAGING MEDIUM COMPRISING LACTATE AND HYPERPOLARISED 13C-PYRUVATE

(76) Inventors: Kevin M. Brindle, Cambridge (GB); Samuel Evan Day, Tucson, AZ (US)

Correspondence Address: GE HEALTHCARE, INC., IP DEPARTMENT 101 CARNEGIE CENTER, PRINCETON, NJ, 08540-6231, US

(21) Appl. No.: 12/376,478

(22) PCT Filed: Aug. 17, 2007
(86) PCT No.: PCT/NO07/00287

§ 371 Date: Feb. 5, 2009

(30) Foreign Application Priority Data
Aug. 18, 2006 (NO) 20063701

(51) Int. Cl. A61K 49/06; C12Q 1/02

(52) U.S. Cl. 424/9.3; 435/29

(57) ABSTRACT

The invention relates to an imaging medium containing lactate and hyperpolarised ¹³C-pyruvate, a method to produce said imaging medium, use of said imaging medium and methods of ¹³C-MR imaging and/or ¹³C-MR spectroscopy wherein said imaging medium is used.

The invention relates to an imaging medium containing lactate and hyperpolarised ¹³C-pyruvate, a method to produce said imaging medium, use of said imaging medium and methods of ¹³C-MR imaging and/or ¹³C-MR spectroscopy wherein said imaging medium is used.

Magnetic resonance (MR) imaging (MRI) is a technique that has become particularly attractive to physicians as images of a patients body or parts thereof can be obtained in a non-invasive way and without exposing the patient and the medical personnel to potentially harmful radiation such as X-rays. Because of its high quality images and good spatial and temporal resolution, MRI is a favourable imaging technique for imaging soft tissue and organs.

MRI may be carried out with or without MR contrast agents. However, contrast-enhanced MRI usually enables the detection of much smaller tissue changes which makes it a powerful tool for the detection of early stage tissue changes like for instance small tumours or metastases.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

Despite the undisputed excellent properties of the aforementioned contrast agents their use is not without any risks. Although paramagnetic metal chelates have usually high stability constants, it is possible that toxic metal ions are released in the body after administration. Further, these type of contrast agents show poor specificity.

WO-A-99/35508 discloses a method of MR investigation of a patient using a hyperpolarised solution of a high T₁ agent as MRI contrast agent. The term “hyperpolarisation” means enhancing the nuclear polarisation of NMR active nuclei present in the high T₁ agent, i.e. nuclei with non-zero nuclear spin, preferably ¹³C- or ¹⁵N-nuclei. Upon enhancing the nuclear polarisation of NMR active nuclei, the population difference between excited and ground nuclear spin states of these nuclei is significantly increased and thereby the MR signal intensity is amplified by a factor of hundred and more. When using a hyperpolarised ¹³C- and/or ¹⁵N-enriched high T₁ agent, there will be essentially no interference from background signals as the natural abundance of ¹³C and/or ¹⁵N is negligible and thus the image contrast will be advantageously high. The main difference between conventional MRI contrast agents and these hyperpolarised high T₁ agents is that in the former changes in contrast are caused by affecting the relaxation times of water protons in the body whereas the latter class of agents can be regarded as non-radioactive tracers, as the signal obtained arises solely from the agent.

A variety of possible high T₁ agents for use as MR imaging agents are disclosed in WO-A-99/35508, including non-endogenous and endogenous compounds like acetate, pyruvate, oxalate or gluconate, sugars like glucose or fructose, urea, amides, amino acids like glutamate, glycine, cysteine or aspartate, nucleotides, vitamins like ascorbic acid, penicillin derivates and sulphonamides. It is further stated that intermediates in metabolic cycles such as the citric acid cycle are preferred imaging agents for MR imaging of metabolic activity.

Hyperpolarised MR imaging agents that play a role in the metabolic processes in the human and non-human animal body are of great interest, as these hyperpolarised imaging agents can be used to get information about the metabolic state of a tissue in an in vivo MR investigation, i.e. they are useful for in vivo imaging of metabolic activity. Information of the metabolic status of a tissue might for instance be used to discriminate between healthy and diseased tissue.

Pyruvate is a compound that plays a role in the citric acid cycle and the conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR studying of metabolic processes in the human body. Hyperpolarised ¹³C-pyruvate may for instance be used as an MR imaging agent for in vivo tumour imaging as described in detail in WO-A-2006/011810 and for assessing the viability of myocardial tissue by MR imaging as described in detail in WO-A-2006/054903.

Pyruvate is an endogenous compound which is very well tolerated by the human body, even in high concentrations. As a precursor in the citric acid cycle, pyruvate plays an important metabolic role in the human body. Pyruvate is converted into different compounds: its transamination results in alanine, via oxidative decarboxylation, pyruvate is converted into acetyl-CoA and carbon dioxide (which is further converted to bicarbonate), the reduction of pyruvate results in lactate and its carboxylation in oxaloacetate.

Further, the metabolic conversion of hyperpolarised ¹³C-pyruvate to its metabolites hyperpolarised ¹³C-lactate, hyperpolarised ¹³C-bicarbonate (in the case of ¹³C₁-pyruvate, ¹³C_{1,2}-pyruvate or ¹³C_{1,2,3}-pyruvate only) and hyperpolarised ¹³C-alanine can be used for in vivo MR study of metabolic processes in the human body. ¹³C₁-pyruvate has a T₁ relaxation in human full blood at 37° C. of about 42 s, however, the conversion of hyperpolarised ¹³C-pyruvate to hyperpolarised ¹³C-lactate, hyperpolarised ¹³C-bicarbonate and hyperpolarised ¹³C-alanine has been found to be fast enough to allow signal detection from the ¹³C-pyruvate parent compound and its metabolites. The amount of alanine, bicarbonate and lactate is dependent on the metabolic status of the tissue under investigation. The MR signal intensity of hyperpolarised ¹³C-lactate, hyperpolarised ¹³C-bicarbonate and hyperpolarised ¹³C-alanine is related to the amount of these compounds and the degree of polarisation left at the time of detection, hence by monitoring the conversion of hyperpolarised ¹³C-pyruvate to hyperpolarised ¹³C-lactate, hyperpolarised ¹³C-bicarbonate and hyperpolarised ¹³C-alanine it is possible to study metabolic processes in vivo in the human or non-human animal body by using non-invasive MR imaging or MR spectroscopy.

The MR signal amplitudes arising from the different pyruvate metabolites vary depending on the tissue type. The unique metabolic peak pattern formed by alanine, lactate, bicarbonate and pyruvate can be used as fingerprint for the metabolic state of the tissue under examination.

It has now been found that an MR imaging agent containing non-hyperpolarised lactate and hyperpolarised ¹³C-pyruvate has superior properties compared to hyperpolarised ¹³C-pyruvate alone. The use of such an imaging agent leads to an increased amount of observable ¹³C-lactate and thus an increased MR signal from ¹³C-lactate. The signal from ¹³C-lactate is the signal one would monitor for MR tumour imaging where tumour tissue is indicated by a high ¹³C-lactate signal as described in WO-A-2006/011810. With an increased signal from ¹³C-lactate, it may be possible to detect smaller tumours or tumour tissue at a very early stage. The signal from ¹³C-lactate is also the signal one would monitor for MR cardiac imaging where myocardial tissue at risk, i.e. ischemic myocardial tissue, is identified by the lowest ¹³C-bicarbonate signal and/or the highest ¹³C-lactate signal as described in WO-A-2006/054903. Further, the signal from ¹³C-lactate is the signal one would monitor for MR imaging of cell death, where dying tissue is indicated by a low or absent ¹³C-lactate signal.

Thus, in a first aspect the invention provides a method of ¹³C-MR imaging or ¹³C-MR spectroscopy using an imaging medium comprising lactate and hyperpolarised ¹³C-pyruvate.

The terms “hyperpolarised” and “polarised” are used interchangeably hereinafter and denote a nuclear polarisation level in excess of 0.1%, more preferred in excess of 1% and most preferred in excess of 10%.

The level of polarisation may for instance be determined by solid state ¹³C-NMR measurements in solid hyperpolarised ¹³C-pyruvate, e.g. solid hyperpolarised ¹³C-pyruvate obtained by dynamic nuclear polarisation (DNP) of ¹³C-pyruvate. The solid state ¹³C-NMR measurement preferably consists of a simple pulse-acquire NMR sequence using a low flip angle. The signal intensity of the hyperpolarised ¹³C-pyruvate in the NMR spectrum is compared with signal intensity of ¹³C-pyruvate in a NMR spectrum acquired before the polarisation process. The level of polarisation is then calculated from the ratio of the signal intensities of before and after polarisation.

In a similar way, the level of polarisation for dissolved hyperpolarised ¹³C-pyruvate may be determined by liquid state NMR measurements. Again the signal intensity of the dissolved hyperpolarised ¹³C-pyruvate is compared with the signal intensity of the dissolved ¹³C-pyruvate before polarisation. The level of polarisation is then calculated from the ratio of the signal intensities of ¹³C-pyruvate before and after polarisation.

The term “imaging medium” denotes a liquid composition comprising hyperpolarised ¹³C-pyruvate as the MR active agent, i.e. imaging agent and lactate which is non-hyperpolarised. The imaging medium according to the invention may be used as imaging medium in MR imaging or as MR spectroscopy agent in MR spectroscopy.

The imaging medium used in the method of the invention may be used as an imaging medium for in vivo ¹³C-MR imaging or spectroscopy, i.e. in living human or non-human animal beings. Further, the imaging medium used in the method of the invention may be used as imaging medium for in vitro ¹³C-MR imaging or spectroscopy, e.g. in cell cultures, samples derived from a human or non human body like for instance urine, saliva or blood or in ex vivo tissue, for instance ex vivo tissue obtained from a biopsy.

The isotopic enrichment of the hyperpolarised ¹³C-pyruvate used in the method of the invention is preferably at least 75%, more preferably at least 80% and especially preferably at least 90%, an isotopic enrichment of over 90% being most preferred. Ideally, the enrichment is 100%. ¹³C-pyruvate used in the method of the invention may be isotopically enriched at the C1-position (in the following denoted ¹³C₁-pyruvate), at the C2-position (in the following denoted ¹³C₂-pyruvate), at the C3-position (in the following denoted ¹³C₃-pyruvate), at the C1- and the C2-position (in the following denoted ¹³C_{1,2}-pyruvate), at the C1- and the C3-position (in the following denoted ¹³C_{1,3}-pyruvate), at the C2- and the C3-position (in the following denoted ¹³C_{2,3}-pyruvate) or at the C1-, C2- and C3-position (in the following denoted ¹³C_{1,2,3}-pyruvate). Isotopic enrichment at the C1-position is preferred since ¹³C₁-pyruvate has a higher T₁ relaxation in human full blood at 37° C. (about 42 s) than ¹³C-pyruvate which is isotopically enriched at other C-positions.

Hyperpolarisation of NMR active ¹³C-nuclei may be achieved by different methods which are for instance described in described in WO-A-98/30918, WO-A-99/24080 and WO-A-99/35508, which are incorporated herein by reference and hyperpolarisation methods are polarisation transfer from a noble gas, “brute force”, spin refrigeration, the parahydrogen method and dynamic nuclear polarisation (DNP).

To obtain hyperpolarised ¹³C-pyurvate, it is preferred to either polarise ¹³C-pyruvate directly or to polarise ¹³C-pyruvic acid and convert the polarised ¹³C-pyruvic acid to polarised ¹³C-pyruvate, e.g. by neutralisation with a base

One suitable way for obtaining hyperpolarised ¹³C-pyruvate is the polarisation transfer from a hyperpolarised noble gas which is described in WO-A-98/30918. Noble gases having non-zero nuclear spin can be hyperpolarised by the use of circularly polarised light. A hyperpolarised noble gas, preferably He or Xe, or a mixture of such gases, may be used to effect hyperpolarisation of ¹³C-nuclei. The hyperpolarised gas may be in the gas phase, it may be dissolved in a liquid/solvent, or the hyperpolarised gas itself may serve as a solvent. Alternatively, the gas may be condensed onto a cooled solid surface and used in this form, or allowed to sublime. Intimate mixing of the hyperpolarised gas with ¹³C-pyruvate or ¹³C-pyruvic acid is preferred. Hence, if ¹³C-pyruvic acid is polarised, which is a liquid at room temperature, the hyperpolarised gas is preferably dissolved in a liquid/solvent or serves as a solvent. If ¹³C pyruvate is polarised, the hyperpolarised gas is preferably dissolved in a liquid/solvent, which also dissolves pyruvate.

Another suitable way for obtaining hyperpolarised ¹³C-pyruvate is that polarisation is imparted to ¹³C-nuclei by thermodynamic equilibration at a very low temperature and high field. Hyperpolarisation compared to the operating field and temperature of the NMR spectrometer is effected by use of a very high field and very low temperature (brute force). The magnetic field strength used should be as high as possible, suitably higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another suitable way for obtaining hyperpolarised ¹³C-pyruvate is the spin refrigeration method. This method covers spin polarisation of a solid compound or system by spin refrigeration polarisation. The system is doped with or intimately mixed with suitable crystalline paramagnetic materials such as Ni²⁺, lanthanide or actinide ions with a symmetry axis of order three or more. The instrumentation is simpler than required for DNP with no need for a uniform magnetic field since no resonance excitation field is applied. The process is carried out by physically rotating the sample around an axis perpendicular to the direction of the magnetic field. The pre-requisite for this method is that the paramagnetic species has a highly anisotropic g-factor. As a result of the sample rotation, the electron paramagnetic resonance will be brought in contact with the nuclear spins, leading to a decrease in the nuclear spin temperature. Sample rotation is carried out until the nuclear spin polarisation has reached a new equilibrium.

In a preferred embodiment, DNP (dynamic nuclear polarisation) is used to obtain hyperpolarised ¹³C-pyruvate. In DNP, polarisation of MR active nuclei in a compound to be polarized is affected by a polarisation agent or so-called DNP agent, a compound comprising unpaired electrons. During the DNP process, energy, normally in the form of microwave radiation, is provided, which will initially excite the DNP agent. Upon decay to the ground state, there is a transfer of polarisation from the unpaired electron of the DNP agent to the NMR active nuclei of the compound to be polarised, e.g. to the ¹³C nuclei in ¹³C-pyruvate. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. The DNP technique is for example further described in WO-A-98/58272 and in WO-A-01/96895, both of which are included by reference herein.

To polarise a compound by the DNP method, a mixture of the compound to be polarised and a DNP agent is prepared (“a sample”) which is then frozen and inserted into a DNP polariser for polarisation. After the polarisation, the frozen solid hyperpolarized sample is rapidly transferred into the liquid state either by melting it or by dissolving it in a suitable dissolution medium. Dissolution is preferred and the dissolution process of a frozen hyperpolarized sample and suitable devices therefore are described in detail in WO-A-02/37132. The melting process and suitable devices for the melting are for instance described in WO-A-02/36005.

In order to obtain a high polarisation level in the compound to be polarised said compound and the DNP agent need to be in intimate contact during the DNP process. This is not the case if the sample crystallizes upon being frozen or cooled. To avoid crystallization, either glass formers need to be present in the sample or compounds need to be chosen for polarisation which do not crystallize upon being frozen but rather form a glass.

As mentioned earlier ¹³C-pyruvic acid or ¹³C-pyruvate are suitable starting materials to obtain hyperpolarized ¹³C-pyruvate.

Isotopically enriched ¹³C-pyruvate is commercially available, e.g. as sodium ¹³C-pyruvate. Alternatively, it may be synthesized as described by S. Anker, J. Biol. Chem. 176, 1948, 133-1335.

Several methods for the synthesis of ¹³C₁-pyruvic acid are known in the art. Briefly, Seebach et al., Journal of Organic Chemistry 40(2), 1975, 231-237 describe a synthetic route that relies on the protection and activation of a carbonyl-containing starting material as an S,S-acetal, e.g. 1,3-dithian or 2-methyl-1,3-dithian. The dithiane is metallated and reacted with a methyl-containing compound and/or ¹³CO₂. By using the appropriate isotopically enriched ¹³C-component as outlined in this reference, it is possible to obtain ¹³C₁-pyruvate, ¹³C₂-pyruvate or ¹³C_{1,2}-pyruvate. The carbonyl function is subsequently liberated by use of conventional methods described in the literature. A different synthetic route starts from acetic acid, which is first converted into acetyl bromide and then reacted with Cu¹³CN. The nitrile obtained is converted into pyruvic acid via the amide (see for instance S. H. Anker et al., J. Biol. Chem. 176 (1948), 1333 or J. E. Thirkettle, Chem. Commun. (1997), 1025). Further, ¹³C-pyruvic acid may be obtained by protonating commercially available sodium ¹³C-pyruvate, e.g. by the method described in U.S. Pat. No. 6,232,497 or by the method described in WO-A-2006/038811.

The hyperpolarisation of ¹³C-pyruvic acid by DNP is described in detail in WO-A1-2006/011809, which is incorporated herein by reference. Briefly, ¹³C-pyruvic acid may be directly used for DNP since it forms a glass when frozen. After DNP, the frozen hyperpolarised ¹³C-pyruvic acid needs to be dissolved and neutralised, i.e. converted to ¹³C-pyruvate. For the conversion, a strong base is needed. Further, since ¹³C-pyruvic acid is a strong acid, a DNP agent needs to be chosen which is stable in this strong acid. A preferred base is sodium hydroxide and conversion of hyperpolarised ¹³C-pyruvic acid with sodium hydroxide results in hyperpolarised sodium ¹³C-pyruvate, which is the preferred ¹³C-pyruvate for an imaging medium which is used for in vivo MR imaging and/or spectroscopy, i.e. MR imaging and/or spectroscopy carried out on living human or non-human animal beings.

Alternatively, ¹³C-pyruvate, i.e. a salt of ¹³C-pyruvic acid can be used for DNP. Preferred salts are those ¹³C-pyruvates which comprise an inorganic cation from the group consisting of NH₄⁺, K⁺, Rb⁺, Cs⁺, Ca²⁺, Sr²⁺ and Ba²⁺, preferably NH₄⁺, K⁺, Rb⁺ or Cs⁺, more preferably K⁺, Rb⁺, Cs⁺ and most preferably Cs⁺, as in detail described in PCT/NO07/00109 and incorporated by reference herein. The synthesis of these preferred ¹³C-pyruvates is disclosed in PCT/NO07/00109 as well. If the hyperpolarized ¹³C-pyruvate is used in an imaging medium for in vivo MR imaging and/or spectroscopy it is preferred to exchange the inorganic cation from the group consisting of NH₄⁺, K⁺, Rb⁺, Cs⁺, Ca²⁺, Sr²⁺ and Ba²⁺ by a physiologically very well tolerable cation like Na⁺ or meglumine. This may be done by methods known in the art like the use of a cation exchange column.

Further preferred salts are ¹³C-pyruvates of an organic amine or amino compound, preferably TRIS-¹³C₁-pyruvate or meglumine-¹³C₁-pyruvate, as in detail described in WO-A-2007/069909 and incorporated by reference herein. The synthesis of these preferred ¹³C-pyruvates is disclosed in WO-A-2007/069909 as well.

If the hyperpolarised ¹³C-pyruvate used in the method of the invention is obtained by DNP, the sample to be polarised comprising ¹³C-pyruvic acid or ¹³C-pyruvate and a DNP agent may further comprise a paramagnetic metal ion. The presence of paramagnetic metal ions in composition to be polarised by DNP has found to result in increased polarisation levels in the ¹³C-pyruvic acid/¹³C-pyruvate as described in detail in WO-A-2007/064226 which is incorporated herein by reference.

As mentioned earlier, the imaging medium according to the method of the invention may be used as imaging medium for in vivo MR imaging and/or spectroscopy, i.e. MR imaging and/or spectroscopy carried out on living human or non-human animal beings. Such an imaging medium preferably comprises in addition to the MR active agent ¹³C-pyruvate an aqueous carrier, preferably a physiologically tolerable and pharmaceutically accepted aqueous carrier like water, a buffer solution or saline. Such an imaging medium may further comprise conventional pharmaceutical or veterinary carriers or excipients, e.g. formulation aids such as are conventional for diagnostic compositions in human or veterinary medicine.

Further, the imaging medium according to the method of the invention may be used as imaging medium for in vitro MR imaging and/or spectroscopy, e.g. for detecting cell death in cell cultures or ex vivo tissues. Such an imaging agent preferably comprises in addition to the MR active agent ¹³C-pyruvate a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

The imaging medium used in the method of the invention contains lactate and hyperpolarised ¹³C-pyruvate. The lactate is non-hyperpolarised. Lactate is suitably added to the hyperpolarised ¹³C-pyruvate after the polarisation process. Several ways of adding the lactate are possible. Where the polarisation process results in a liquid composition comprising the hyperpolarised ¹³C-pyruvate, lactate may be dissolved in said liquid composition or a solution of lactate in a suitable solvent, preferably an aqueous carrier may be added to the liquid composition. If the polarisation process results in a solid composition comprising the hyperpolarised ¹³C-pyruvate, lactate may be dissolved in the dissolution medium which is used to dissolve the solid composition. For instance ¹³C-pyruvate polarised by the DNP method may be dissolved in an aqueous carrier like water or a buffer solution containing lactate. If hyperpolarised ¹³C-pyruvate has been obtained by dynamic nuclear polarisation, it is preferred to add lactate to the final liquid composition, i.e. to the liquid composition after dissolution/melting or to the liquid composition after removal of the DNP agent and/or an optional paramagnetic metal ion. Again the lactate may be added as a solid to the liquid composition or preferably dissolved in a suitable solvent, e.g. an aqueous carrier like water or a buffer solution. To promote dissolution of the lactate, several means known in the art, such as stirring, vortexing or sonication may be used. However, methods are preferred which are quick and do not require a mixing device or help coming into contact with the liquid composition. Methods like vortexing or sonication are thus preferred.

Suitably, lactate in added in the form of lactic acid or a salt of lactic acid, preferably lithium lactate or sodium lactate, most preferably sodium lactate. The concentration of hyperpolarised ¹³C-pyruvate and lactate in the imaging medium used in the method of the invention is about equal or equal or lactate is present at a lower or higher concentration than ¹³C-pyruvate. If for instance the imaging agent contains x M ¹³C-pyruvate, it contains x M or about x M or less lactate but preferably not less than a tenth of x M lactate or more lactate but preferably not more than three times x M lactate. In a preferred embodiment, the concentration of lactate in the imaging medium used in the method of the invention is about equal or equal to the concentration of hyperpolarised ¹³C-pyruvate. The term “about equal concentration” denotes a lactate concentration which is +/−30% of the concentration of ¹³C-pyruvate, preferably +/−20%, more preferably +/−10%.

To be used as an imaging medium for in vivo ¹³C-MR imaging or ¹³C-MR spectroscopy in the method of the invention, the imaging medium comprising lactate and hyperpolarised ¹³C-pyruvate is provided as a composition that is suitable for administration to a living human or non-human animal body. The imaging medium preferably comprises an aqueous carrier like a buffer or a mixture of buffers as described above. The imaging medium may further comprise conventional pharmaceutically acceptable carriers, excipients and formulation aids. Thus, the imaging medium may for example include stabilizers, osmolality adjusting agents, solubilising agents and the like.

If the imaging medium used in the method of the invention is used for in vivo MR imaging or spectroscopy, e.g. in a living human or non-human animal body, said imaging medium is preferably administered to said body parenterally, preferably intravenously. Generally, the body under examination is positioned in an MR magnet. Dedicated ¹³C-MR RF-coils are positioned to cover the area of interest. Dosage and concentration of the imaging medium will depend upon a range of factors such as toxicity and the administration route. Suitably, the imaging medium is administered in a concentration of up to 1 mmol ¹³C-pyruvate per kg bodyweight, preferably 0.01 to 0.5 mmol/kg, and more preferably 0.1 to 0.3 mmol/kg. The administration rate is preferably less than 10 ml/s, more preferably less than 6 ml/min and most preferable of from 5 ml/s to 0.1 ml/s. At less than 400 s after the administration, preferably less than 120 s, more preferably less than 60 s after the administration, especially preferably 20 to 50 s an MR imaging sequence is applied that encodes the volume of interest in a combined frequency and spatial selective way. This will result in metabolic images of ¹³C-pyruvate and ¹³C-lactate. The exact time of applying an MR sequence is highly dependent on the volume of interest.

To be used as an imaging medium for in vitro ¹³C-MR imaging or ¹³C-MR spectroscopy in the method of the invention, the imaging medium comprising lactate and hyperpolarised ¹³C-pyruvate is provided as a composition that is suitable for being added to, for instance, cell cultures, samples derived from a human or non human body or ex vivo tissues like biopsy tissues. As it is apparent for the skilled person, pharmaceutically acceptable carriers, excipients and formulation aids may be present in the imaging medium but are not required to be present for such a purpose, and the imaging medium thus preferably comprises an aqueous carrier like a buffer or a mixture of buffers as described above and/or one or more non aqueous solvents compatible with cell cultures or tissue like DMSO or methanol. An imaging medium for use in cell cultures, samples derived from a human or non-human body or ex vivo tissue like biopsy tissues is preferably 10 mM to 100 mM in ¹³C-pyruvate, more preferably 20 mM to 90 mM and most preferably 40 to 80 mM in ¹³C-pyruvate.

Viewed from a further aspect, the invention provides an imaging medium comprising lactate and hyperpolarised ¹³C-pyruvate.

In a further preferred embodiment, the imaging medium according to the invention contains about equal or equal concentrations of lactate and hyperpolarised ¹³C-pyruvate or contains lactate in a lower or higher concentration than ¹³C-pyruvate. If for instance the imaging agent contains x M ¹³C-pyruvate, it contains x M or about x M or less lactate but preferably not less than a tenth of x M lactate or more lactate but preferably not more than three times x M lactate. In a preferred embodiment, the concentration of lactate in the imaging medium according to the invention is about equal or equal to the concentration of hyperpolarised ¹³C-pyruvate. The term “about equal concentration” denotes a lactate concentration which is +/−30% of the concentration of ¹³C-pyruvate, preferably +/−20%, more preferably +/−10%.

In another preferred embodiment, the lactate is selected from the group consisting of lactic acid, lithium lactate or sodium lactate.

The imaging medium is preferably used in ¹³C-MR imaging or ¹³C-MR spectroscopy.

If the imaging medium according to the invention is used as in vivo imaging medium, i.e. administered to a living human or non-human animal being, said imaging medium preferably further an aqueous carrier like a buffer or a mixture of buffers as described above. The imaging medium may further comprise conventional pharmaceutically acceptable carriers, excipients and formulation aids. Thus, the imaging medium may for example include stabilizers, osmolality adjusting agents, solubilising agents and the like.

If the imaging medium according to the invention is used for in vitro ¹³C-MR imaging or ¹³C-MR spectroscopy it may further comprise pharmaceutically acceptable carriers, excipients and formulation aids as mentioned in the previous paragraph. However, it is apparent for the skilled person that such pharmaceutically acceptable carriers, excipients and formulation aids are not required to be present for such a purpose. The imaging medium thus preferably further comprises an aqueous carrier like a buffer or a mixture of buffers as described above and/or one or more non aqueous solvents compatible with cell cultures or tissue like DMSO or methanol.

Yet another aspect of the invention is a method for producing an imaging medium comprising lactate and hyperpolarised ¹³C-pyruvate, wherein the hyperpolarised ¹³C-pyruvate is obtained by dynamic nuclear polarisation of ¹³C-pyruvic acid or ¹³C-pyruvate and lactate is added to a solution of said hyperpolarised ¹³C-pyruvate.

A further aspect of the invention is the use of an imaging medium according to the invention for in vivo study of metabolic processes in the human or non human animal body using ¹³C-MR imaging and/or ¹³C-MR spectroscopy.

A further aspect of the invention is the use of an imaging medium according to the invention for in vitro study of metabolic processes in cell cultures, samples derived from a human or non human body or ex vivo tissue using ¹³C-MR imaging and/or ¹³C-MR spectroscopy.

Yet another aspect of the invention is the use of an imaging medium according to the invention for in vivo identification of tumour tissue in the human or non human animal body using ¹³C-MR imaging and/or ¹³C-MR spectroscopy.

Yet another aspect of the invention is the use of an imaging medium according to the invention for in vitro identification of tumour cells in a cell culture, in samples derived from a human or non human body or in ex vivo tissue using ¹³C-MR imaging and/or ¹³C-MR spectroscopy.

Suitable ¹³C-MR imaging and/or ¹³C-MR spectroscopy protocols for in vivo identification of tumour tissue and in vitro identification of tumour cells are described in WO-A-2006/011810.

Yet another aspect of the invention is the use of an imaging medium according to the invention for in vivo assessment of the viability of myocardial tissue in the human or non human animal body using ¹³C-MR imaging and/or ¹³C-MR spectroscopy.

Suitable ¹³C-MR imaging and/or ¹³C-MR spectroscopy protocols for in vivo assessment of the viability of myocardial tissue in the human or non human animal body are described in WO-A-2006/054903.

Yet another aspect of the invention is the use of an imaging medium according to the invention for in vivo detection of cell death in the human or non human animal body using ¹³C-MR imaging and/or ¹³C-MR spectroscopy.

Yet another aspect of the invention is the use of an imaging medium according to the invention for in vitro detection of cell death in a cell culture, in samples derived from a human or non human body or in ex vivo tissue using ¹³C-MR imaging and/or ¹³C-MR spectroscopy.

Cell death (e.g. apoptosis and necrosis) can be detected by the method of the invention by following the ¹³C-pyruvate signal and the signal of its metabolite ¹³C-lactate over time. In viable cells the ¹³C-pyruvate signal decays over time. The ¹³C-lactate signal increases first due to metabolic conversion of ¹³C-pyruvate to ¹³C-lactate and then slowly decreases mainly due to relaxation. In dying cells, the metabolic conversion of ¹³C-pyruvate to ¹³C-lactate is greatly decreased and although the ¹³C-pyruvate signal decays over time, the ¹³C-lactate signal only increases slightly or may not be detectable at all, depending on the degree of cell death/amount of dying/dead cells. Without wanting to be bound to this theory, we believe that this is either due to the loss of activity of the enzyme lactate dehydrogenase which catalyses the interconversion of pyruvate and lactate with concomitant interconversion of NADH and NAD⁺ and/or due to the loss of the co-factors NADH and NAD⁺ and/or due to a decrease in the cellular lactate concentration.

³¹P-NMR measurements on acid-extracts of EL-4 murine lymphoma cells induced to undergo cell death by treatment with etoposide have demonstrated a decrease in the intensity of the resonances from NAD(H), when compared to untreated control cells. Loss of NAD(H) can be explained by DNA-damage induced activation of the enzyme poly-ADP-ribose polymerase (PARP), which polyadenylates various proteins and uses NAD⁺ as a substrate. There was also an increase in the resonances from the glycolytic intermediate, fructose-1,6-bisphosphate (FBP), which can be explained by inhibition of the glycolytic enzyme, glyceraldehyde 3-phosphate dehydrogenase (GAPDH), through loss of its coenzyme NAD(H). Inhibition of PARP activity with a competitive inhibitor (20 mM nicotinamide) or 3-aminobenzamide (10 mM) inhibited the loss of NAD(H) and the increase in FBP concentration observed in apoptotic cells.

This loss of NAD(H) due to PARP activation should also inhibit lactate dehydrogenase (LDH) activity and hence the measured flux of ¹³C-label between pyruvate and lactate. Consistent with this hypothesis is the observation that cells treated with both nicotinamide and etoposide or 3-aminobenzamide and etoposide maintained their ability to transfer the hyperpolarised ¹³C-label between the pyruvate and lactate pools, while still exhibiting the common morphological features of dying cells, as detected using fluorescence microscopy.

If the imaging medium according to the invention is used for the detection of cell death in vitro e.g. for the detection of cell death in a cell culture, in samples derived from a human or non human body or in ex vivo tissue the imaging medium is 10 mM to 50 mM in ¹³C-pyruvate, preferably 20 mM to 40 mM.

If the imaging medium according to the invention is used for the detection of cell death in vivo, i.e. for the detection cell death in a living human or non-human animal body, the imaging medium according to the invention is preferably administered to said body parenterally, preferably intravenously. Generally, the body under examination is positioned in the MR magnet. Dedicated ¹³C-MR RF-coils are positioned to cover the area of interest. Dosage and concentration of the imaging medium will depend upon a range of factors such as toxicity and the administration route. Suitably, the imaging medium is administered in a concentration of up to 1 mmol ¹³C-pyruvate per kg bodyweight, preferably 0.01 to 0.5 mmol/kg, more preferably 0.1 to 0.3 mmol/kg. The administration rate is preferably less than 10 ml/s, more preferably less than 6 ml/min and most preferable of from 5 ml/s to 0.1 ml/s. At less than 400 s after the administration, preferably less than 120 s, more preferably less than 60 s after the administration, especially preferably 20 to 50 s an MR imaging sequence is applied that encodes the volume of interest in a combined frequency and spatial selective way. This will result in metabolic images/spectra of ¹³C-pyruvate and ¹³C-lactate. The exact time of applying an MR sequence is highly dependent on the volume of interest for detecting apoptosis.

An MR imaging sequence is applied that encodes the volume of interest in a combined frequency and spatially selective way and the ¹³C-MR signal of ¹³C-pyruvate is followed by MR imaging or spectroscopy over a time period from the addition of the imaging agent (t=0) to about 10 min, preferably 6 min and more preferably 5 min. In the same time period, the appearance, increase and subsequent decrease of the ¹³C-lactate signal is monitored. To get a quantitative assessment, MR imaging or spectroscopy of healthy cells or tissue may carried out and the results—i.e. the amount of lactate formed over a given time period—may be compared.

The encoding of the volume of interest can be achieved by using so-called spectroscopic imaging sequences as described in for instance T. R. Brown et al., Proc. Natl. Acad. Sci. USA 79, 3523-3526 (1982); A. A. Maudsley, et al., J. Magn. Res 51, 147-152 (1983). Spectroscopic image data contain a number of volume elements in which each element contains a full ¹³C-MR spectrum. ¹³C-pyruvate and its metabolite ¹³C-lactate have their unique position in a ¹³C-MR spectrum and their resonance frequency can be used to identify them. The integral of the peak at its resonance frequency is directly related to the amount of ¹³C-pyruvate and ¹³C-lactate, respectively. When the amount of ¹³C-pyruvate and ¹³C-lactate is estimated using time domain fitting routines as described for instance in L. Vanhamme et al., J Magn Reson 129, 35-43 (1997), images can be generated for ¹³C-pyruvate and 13C-lactate in which a colour coding or grey coding is representative for the amount of ¹³C-pyruvate and ¹³C-lactate measured.

Although spectroscopic imaging methods have proven their value in producing metabolic images using all kinds of MR nuclei e.g. ¹H, ³¹P, ²³Na, the amount of repetitions needed to fully encode the spectroscopic image makes this approach less suitable for hyperpolarised ¹³C. Care has to be taken to ensure hyperpolarised ¹³C-signal is available during the whole MR data acquisition. At the expense of a reduced signal to noise, this can be achieved by reducing the RF-pulse angle that is applied in every phase encoding step. Higher matrix sizes require more phase encoding steps and longer scan times.

Imaging methods based on the pioneering work of P. C. Lauterbur (Nature, 242, 190-191, (1973) and P. Mansfield (J. Phys. C. 6, L422-L426 (1973)), which apply a readout gradient during the data acquisition, will allow for higher signal to noise images or the equivalent, higher spatial resolution images. However, these imaging methods in their basic form will not be able to produce separate images for ¹³C-pyruvate and ¹³C-lactate, i.e. the identification of specific metabolites is not possible.

In a preferred embodiment, imaging sequences are used that will make use of multi-echoes to code for the frequency information. Sequences that can produce separate water and fat ¹H-images are for example described in G. Glover, J Magn Reson Imaging 1991; 1:521-530 and S. B. Reeder et al., MRM 51 35-45 (2004). Since the metabolites to be detected and as such their MR frequencies are known, the approach discussed in the references above can be applied to acquire direct images of ¹³C-pyruvate and ¹³C-lactate. This procedure makes more efficient use of the hyperpolarised ¹³C-MR signal, giving a better signal quality compared to spectroscopic imaging, a higher spatial resolution and faster acquisition times.

In a preferred embodiment, the detection of cell death comprises acquiring direct ¹³C-MR images or spectra of ¹³C-pyruvate and ¹³C-lactate from a human or non-human animal body pre-administered with the imaging medium according to the invention or from a cell culture, samples derived from a human or non human body or in ex vivo tissue where the imaging medium according to the invention has been added to. Cell death is identified and detected by a low ¹³C-signal intensity from ¹³C-lactate or an absent signal from ¹³C-lactate or a decreased rate of formation of ¹³C-lactate.

To correct for the pyruvate signal, both lactate and pyruvate images may be normalized to the maximum value in each individual image. Second, the normalized lactate image is multiplied by the inverted pyruvate image, e.g. the maximum pyruvate signal in the image minus the pyruvate level for every pixel. As a last step, the intermediate result gained in the operation above is multiplied by the original lactate image. Alternatively, the pyruvate and lactate peak intensities in each pixel of their respective images can be fit to a kinetic model of the flux of ¹³C label between pyruvate and lactate to obtain rate constants for label flux and the spin lattice relaxation times. Correction may need to be made for the effect of multiple RF pulses on the loss of polarization.

Anatomical and/or perfusion information may be included in the detection of cell death if the method is used for detection of cell death in vivo. Anatomical information may for instance be obtained by acquiring a proton or ¹³C-MR image with or without employing a suitable contrast agent. Relative perfusion can be determined by using an MR contrast agent like for instance Omniscan™. Likewise, MR imaging techniques for perfusion measurement without the administration of a contrast agent are known in the art. In a preferred embodiment, a non-metabolised hyperpolarised ¹³C-contrast agent is used to determine quantitative perfusion. Suitable techniques and contrast agents are for instance described in WO-A-O-02/23209. In a more preferred embodiment, hyperpolarised ¹³C-pyruvate is used to determine quantitative perfusion.

In another preferred embodiment, the imaging medium according to the invention is administered repeatedly, thus allowing dynamic studies. Due to the low toxicity of pyruvate and its favourable safety profile, repeated doses of this compound are well tolerated by the patient.

The results obtained do for instance allow the physician to choose the appropriate treatment for the patient under examination or allows the physician to determine whether treatment is successful.

### BRIEF DESCRIPTION OF THE DRAWINGS

FIG. 1 depicts peak intensities of ¹³C₁-pyruvate and ¹³C₁-lactate in an EL4 cell suspension treated with etoposide and in an untreated EL4 cell suspension vs. time. Curve numberings in FIG. 1 denote the following:
1: ¹³C₁-pyruvate intensities (divided by 100) in untreated control cell and etoposide treated cell suspensions
2: ¹³C₁-lactate intensities in a control cell suspension
3: ¹³C₁-lactate intensities in an etoposide treated cell suspension

FIG. 2 shows the effect of etoposide and etoposide/nicotinamide treatment on EL4 cells to the cell death inducing drug etoposide. Bar graphs in FIG. 2 represent 3 experiments +/−standard deviation.

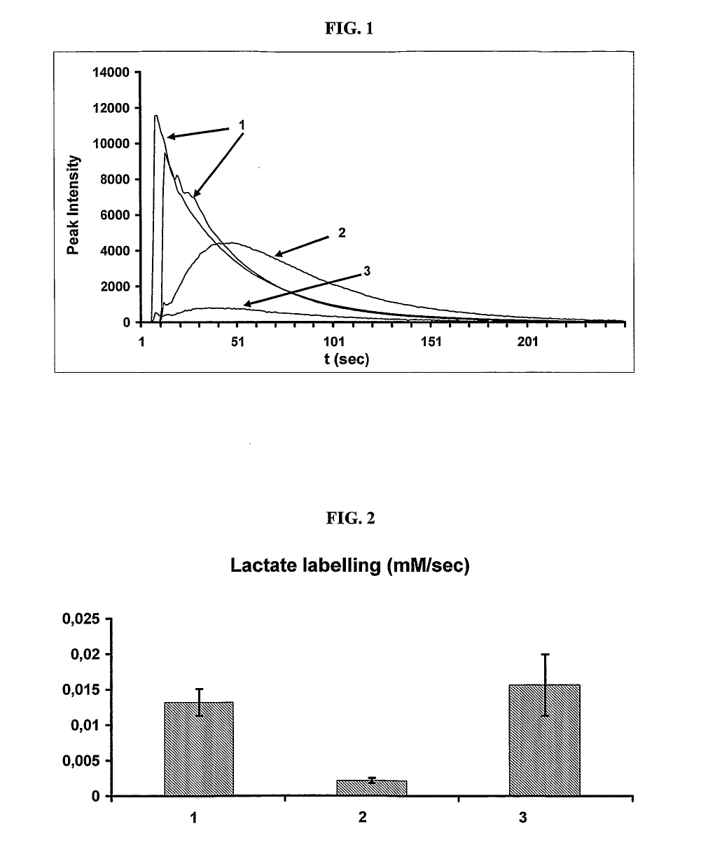

Bar numbering in FIG. 2 denote the following:

1: untreated EL4 cell suspension
2: etoposide treated EL4 cell suspension
3: etoposide/nicotinamide treated EL4 cell suspension

### EXAMPLES

In the following the terms pyruvate, ¹³C-pyruvate and ¹³C₁-pyruvate are used interchangeably and all denote ¹³C₁-pyruvate. Likewise the terms pyruvic acid, ¹³C-pyruvic acid and ¹³C₁-pyruvic acid are used interchangeably and all denote ¹³C₁-pyruvic acid.

### Example 1

### Synthesis of Tris(8-carboxy-2,2,6,6-(tetra(methoxyethyl)benzo-[1,2-4,5′]bis-(1,3)dithiole-4-yl)methyl sodium salt, a DNP agent

10 g (70 mmol) Tris(8-carboxy-2,2,6,6-(tetra(hydroxyethyl) benzo-[1,2-4,5′]-bis-(1,3)-dithiole-4-yl)methyl sodium salt which had been synthesized according to Example 7 of WO-A1-98/39277 were suspended in 280 ml dimethylacetamide under an argon atmosphere. Sodium hydride (2.75 g) followed by methyl iodide (5.2 ml) was added and the reaction which is slightly exothermic was allowed to proceed for 1 hour in a 34° C. water bath for 60 min. The addition of sodium hydride and methyl iodide was repeated twice with the same amounts of each of the compounds and after the final addition, the mixture was stirred at room temperature for 68 hours and then poured into 500 ml water. The pH was adjusted to pH>13 using 40 ml of 1 M NaOH (aq) and the mixture was stirred at ambient temperature for 15 hours to hydrolyse the formed methyl esters. The mixture was then acidified using 50 ml 2 M HCl (aq) to a pH of about 2 and 3 times extracted the ethyl acetate (500 ml and 2×200 ml). The combined organic phase was dried over Na₂SO₄ and then evaporated to dryness. The crude product (24 g) was purified by preparative HPLC using acetonitrile/water as eluents. The collected fractions were evaporated to remove acetonitrile. The remaining water phase was extracted with ethyl acetate and the organic phase was dried over Na₂SO₄ and then evaporated to dryness. Water (200 ml) was added to the residue and the pH was carefully adjusted with 0.1 M NaOH (aq) to 7, the residue slowly dissolving during this process. After neutralization, the aqueous solution was freeze dried.

### Example 2

### Preparation of an Imaging Medium Comprising Lactate and Hyperpolarised ¹³C₁-pyruvate

A 15 mM solution was prepared by dissolving the radical of Example 1 in ¹³C₁-pyruvic acid (44 mg, 91%). The sample was mixed to homogeneity and the solution was placed in a sample cup and inserted in the DNP polariser.

The sample was polarised under DNP conditions at 1.2 K in a 3.35 T magnetic field under irradiation with microwave (94 GHz and 100 mW, respectively). Polarisation was followed by solid state NMR. After 90 min hyperpolarisation, the sample was dissolved in 6 ml of an aqueous solution of 94 mM NaOH, 30 mM NaCl, 40 mM HEPES and 50 mg/litre EDTA. The pH of the dissolved sample was 7.4 with a final ¹³C₁-pyruvate concentration of 75 mM.

2 ml of the resulting solution were combined with 500 μl water containing 18 mg lithium lactate to result in an imaging medium comprising 60 mM hyperpolarised ¹³C₁-pyruvate and 75 mM lactate.

### Example 3

### Detection of Cell Death in Cell Cultures

### 3.1 Preparation of EL4 Cells

EL4 murine lymphoma cells (10⁸ cells) were treated with 15 μM etoposide (PCH Pharmachemie BV, Harleem), a compound which is known to induce cell death after 16 h exposure. A separate set of cells was treated for 16 hours with 15 μM etoposide plus 20 mM nicotinamide, a known PARP inhibitor. Cell death (apoptosis and necrosis) was confirmed by acridine orange and propidium iodide staining. The cells were washed 3 times with RPMI 1640 growth medium containing 10% FCS at 37° C. and to 2 ml of the etoposide- and etoposide/nicotinamide treated EL4 cell suspension 2 ml of the imaging medium according to Example 2 were added. The final cell suspension thus contained 30 mM hyperpolarised ¹³C₁-pyruvate and 37.5 mM lactate.

### 3.2 ¹³C-MR Spectroscopy of EL4 Cells

¹³C-signal intensities from ¹³C-pyruvate and ¹³C-lactate in etoposide-treated EL4 cell suspensions as described in 3.1 were followed over a time period of 240 seconds from the time of addition of the imaging medium. One ¹³C spectrum per second was acquired using low flip angle pulses at 9.4 T for a total of 240 spectra. A control of non-etoposide treated (untreated) EL4 lymphoma cells was also examined, as outlined above, and the peak intensities of ¹³C-pyruvate and ¹³C-lactate from the untreated and the etoposide were plotted on a graph (FIG. 1).

### 3.3 ¹³C-MR Spectroscopy of EL4 Cells

¹³C-signal intensities from ¹³C-pyruvate and ¹³C-lactate in etoposide-treated and etoposide/nicotinamide treated EL4 cell suspensions as described in 3.1 were followed over a time period of 240 seconds from the time of addition of the imaging medium. One ¹³C spectrum per second was acquired using low flip angle pulses at 9.4 T for a total of 240 spectra. A control of non-etoposide treated (untreated) EL4 lymphoma cells was also examined, as outlined above, and the peak intensities of ¹³C-pyruvate and ¹³C-lactate from the untreated, the etoposide treated and the etoposide/nicotinamide treated EL4 cells were compared. The data were fit to a two-site exchange model based on the modified Bloch equations, and the rate constants for the forward and reverse exchange ¹³C-fluxes were determined. Bar graphs in FIG. 2 represent 3 experiments +/−standard deviation.

What is claimed is:

1. An imaging medium comprising lactate and hyperpolarised ¹³C-pyruvate.

2. The imaging medium according to claim 1 containing about equal or equal concentrations of lactate and hyperpolarised ¹³C-pyruvate.

3. The imaging medium according to claim 1 wherein the lactate is selected from lactic acid, sodium lactate or lithium lactate.

4. The imaging medium according to claim 1, wherein the imaging medium further comprises an aqueous carrier.

5. The imaging medium according to claim 1, wherein the imaging medium further comprises conventional pharmaceutically acceptable carriers and/or excipients and/or formulation aids.

6. The imaging medium according to claim 1 for use in in vivo ¹³C-MR imaging and/or ¹³C-MR spectroscopy.

7. The imaging medium according to claim 1, wherein the imaging medium further comprises one or more non-aqueous solvents, preferably DMSO and/or methanol.

8. The imaging medium according to claim 1 for use in in vitro ¹³C-MR imaging and/or ¹³C-MR spectroscopy.

9. Use of the imaging medium according to claim 1 for in vivo study of metabolic processes in the human or non human animal body using ¹³C-MR imaging and/or ¹³C-MR spectroscopy.

10. Use of the imaging medium according to claim 1 for in vivo identification of tumour tissue in the human or non human animal body using ¹³C-MR imaging and/or ¹³C-MR spectroscopy.

11. Use of the imaging medium according to claim 1 for in vivo assessment of the viability of myocardial tissue in the human or non human animal body using ¹³C-MR imaging and/or ¹³C-MR spectroscopy.

12. Use of the imaging medium according to claim 1 for in vivo detection of cell death in the human or non-human animal body using ¹³C-MR imaging and/or ¹³C-MR spectroscopy.

13. Use of the imaging medium according to claim 7 for in vitro study of metabolic processes in cell cultures, samples derived from a human or non human body or ex vivo tissue using ¹³C-MR imaging and/or ¹³C-MR spectroscopy.

14. Use of the imaging medium according to claim 7 for in vitro identification of tumour cells in a cell culture, samples derived from a human or non human body or ex vivo tissue using ¹³C-MR imaging and/or ¹³C-MR spectroscopy.

15. Use of the imaging medium according to claim 7 for in vitro detection of cell death in a cell culture, samples derived from a human or non human body or ex vivo tissue using ¹³C-MR imaging and/or ¹³C-MR spectroscopy.

16. Method for producing an imaging medium according to claim 1, wherein the hyperpolarised ¹³C-pyruvate is obtained by dynamic nuclear polarisation of ¹³C-pyruvic acid or ¹³C-pyruvate and lactate is added to a solution of said hyperpolarised ¹³C-pyruvate.

17. Method of ¹³C-MR imaging and/or ¹³C-MR spectroscopy using an imaging medium according to claim 1.
